(19) United States
(12) Patent Application Publication
(10) Pub. No.: US 2025/0389688 A1
(43) Pub. Date: Dec. 25, 2025

(54) REGULATING THE ELECTROCHEMICAL REDOX BEHAVIOR OF SEMICONDUCTING POLYMERS ON SOFT AND STRETCHABLE SUBSTRATES AND METHODS OF USE

(71) Applicant: The University of Hong Kong, Hong Kong (HK)

(72) Inventors: Shiming ZHANG, Hong Kong (HK); Dingyao LIU, Hong Kong (HK); Xinyu TIAN, Hong Kong (HK); Jing BAI, Hong Kong (HK)

(21) Appl. No.: 18/879,269

(22) PCT Filed: Jun. 17, 2023
(86) PCT No.: PCT/CN2023/100893

§ 371 Date: Dec. 27, 2024

Related U.S. Application Data
(60) Provisional application No. 63/367,160, filed on Jun. 28, 2022.

(51) Int. Cl. G01N 27/327; A61B 5/00; A61B 5/268; G01N 27/414

(52) U.S. Cl. CPC G01N 27/3277; A61B 5/268; A61B 5/6801; G01N 27/4145; A61B 2562/125; A61B 2562/164

(57) ABSTRACT

An intrinsically stretchable organic electrochemical transistor with overall performance benchmarkable to a rigid device. The high performance was realized by reducing the oxygen level (P_{O2}) of the stretchable substrates, which facilitates the de-doping of the conducting polymer channel. The high-performance intrinsically stretchable OECT is usable not only as a new device paradigm to impact the field of soft bioelectronics and promote the use of tissue-like stretchable OECTs in areas such as epidermal biosensing, soft neuromorphic computing and soft human-machine interfaces, but also to reveal a new critical parameter to alter the performance of stretchable conducting polymer-based devices.

### CROSS-REFERENCE TO RELATED APPLICATIONS

The present application claims priority from a U.S. provisional patent application Ser. No. 63/367,160 filed 28 Jun. 2022, and the disclosures of which are incorporated by reference in their entirety.

### TECHNICAL FIELD

The present invention relates to a method to modify the electrochemical behavior of conducting polymers and conducting polymer-made devices on soft and stretchable substrates. More specifically, the present invention introduces stretchable devices equipped with an intrinsically stretchable organic electrochemical transistor modified with the above method, which displays performance benchmarkable to rigid devices.

### BACKGROUND OF THE INVENTION

Organic electrochemical devices, based on semiconducting polymer thin films, stand out due to their working mechanism, which closely resembles that of a real neuron, both being organic, ionic-signaling, and electrochemically operated. To further promote the application of organic synapses at soft biological interfaces, stretchable devices have been proposed to minimize mechanical mismatch. A major challenge for the development of stretchable organic electrochemical devices is that material systems and fabrication methods need to be systemically rebuilt.

For example, in 2017, Zhang et al. reported the first fully stretchable organic electrochemical transistor (OECT) on an elastic polydimethylsiloxane (PDMS) substrate [1]. The device was fabricated by using a buckling method and a solid-state hydrogel as the stretchable electrolyte. A 10*10 stretchable OECT array was demonstrated. The stretchable OECTs could withstand a strain up to 30% with stable performance. In 2018, Ramuz et al. reported stretchable OECTs by using a laser etching method to pattern stretchable serpentine electrodes and channels [2]. The device could maintain a high transconductance of 0.35 mS under a strain of 38%. In 2018, Lee et al. fabricated stretchable OECTs with a stretchable nano-mesh architecture [3]. The device was used for conformal electrocardiogram recording in a living rat. In 2019, Zhang et al. reported the first intrinsically stretchable OECTs by using an ultrathin and microcracked gold film as stretchable interconnect and a microcracked PEDOT:PSS film as a stretchable channel [4]. Matsuhisa et al. reported the use of intrinsically stretchable OECTs for fabricating intrinsically stretchable synaptic transistors [5]. In 2019, Li et al reported stretchable OECTs for glucose detection [6]. The stretchable OECTs sensor maintained its sensing function between 0% and 30% strain. In 2021, Nguyen et al. demonstrated an artificial synapse with a stretchable OECT, where the synaptic enhancement can be controlled by regulating the dynamics of ion transport [7].

Despite the above achievements in advancing stretchable OECTs, the performance of intrinsically stretchable OECTs remains considerably lower compared to their rigid counterparts. For example, the on/off ratio of intrinsically stretchable OECTs fabricated on stretchable substrates such as PDMS were two orders of magnitude lower than that of a rigid device. Further, the charge carrier mobility of stretchable OECTs has yet to be investigated. Recent efforts have been focused on resolving these problems, including introducing buffer layers between substrates and channel materials and using different substrate materials. However, limited progress has been made to identify the critical parameter leading to this phenomenon. Thus, there is a need to clarify the underlying mechanism and provide guidance for future device design.

### SUMMARY OF THE INVENTION

Therefore, provided herein is a method for tuning the electrochemical and electrochromic properties, and the non-linearity and synaptic behaviors of organic electrochemical devices through altering a critical parameter, the oxygen permeability of a stretchable substrate, which significantly impacts the redox behaviors in semiconductors. Taking stretchable OECT as an example, by employing stretchable substrates with low oxygen permeabilities, the on/off ratio was elevated from ˜10 to a value of ˜10⁴, with a high mobility of ˜1.1 cm²V⁻¹s⁻¹; further, the device functions even after cyclic strain tests between 0% and 50%. The devices show high mobility, comparable to that of rigid device, and has potential applications such as electronic skin, soft implantables and soft neuromorphic computing.

In one aspect, a method for modifying the electrochemical properties, electrochromical properties, non-linearity and synaptic behaviors of an electronic device by providing a stretchable substrate selected from one or more of elastomers, hydrogels or hybrid organic-inorganic materials, the stretchable substrate having a selected oxygen permeability of 0.1-50 Barrer; and forming a stretchable redox-active layer on the substrate, the redox-active layer including one or more of conductive polymers, organic molecules or hybrid organic-inorganic molecules.

In an embodiment, the ratio of the poly(3,4-ethylenedioxythiophene) and poly(styrene-sulfonate) in the mixture forming the electrodes and channel is 1:1 to 1:3.

In another embodiment, the method further includes depositing one or more electrodes on the stretchable redox-active layer.

In yet another embodiment the electronic device is a bioelectronic device in the form of a bioelectrode, a wearable biosensor or a bioelectronic implant.

In other embodiment, the electronic device has a shape of micro-wires, macro-wires, micro-mesh, macro-mesh, film, micro-3D structure and/or macro-3D structure.

In yet other embodiment, the stretchable substrate is selected from styrene-butadiene rubber, ethylene propylene diene monomer, poly(styrene-ethylene-butylene-styrene), ethylene-vinyl acrylate and/or thermoplastic polyurethane.

In another aspect, a stretchable device equipped with an intrinsically stretchable organic electrochemical transistor is provided. The device comprises an OECT comprising a stretchable planar gate electrode, source electrode, drain electrode and channel each comprising a conducting polymer; a stretchable ionic gel as a solid-state electrolyte cast on the channel and gate; and a stretchable elastomer substrate. The width/length ratio of the OECT is 3.5 to 50; the on/off ratio of the device is at least 10³; the mobility of the device is at least 0.8 cm²V⁻¹s⁻¹; and the current loss of the transistor is less than 10% when the device is stretched up to 150% of its original length.

In an embodiment, the stretchable substrate is selected from an elastomer, a hydrogel or a hybrid organic-inorganic stretchable polymer.

In a further embodiment, the stretchable substrate is selected from styrene-butadiene rubber, ethylene propylene diene monomer, poly(styrene-ethylene-butylene-styrene), ethylene-vinyl acrylate and/or thermoplastic polyurethane.

In other embodiment, the device has a shape of micro-wires, macro-wires, micro-mesh, macro-mesh, film, micro-3D structure and/or macro-3D structure.

In an embodiment, the conducting polymer is a mixture of poly(3,4-ethylenedioxythiophene) and poly(styrene-sulfonate).

In another embodiment, the ratio of the poly(3,4-ethylenedioxythiophene) to poly(styrene-sulfonate) is 1:1 to 1:3.

In another embodiment, the stretchable ionic gel is a polyacrylamide-based gel.

In yet other embodiment, the reduction of the mobility of the transistor is less than 10% when the device is stretched up to 110% of its original length.

In yet another embodiment, the reduction of the mobility of the transistor is less than 25% when the device is stretched up to 150% of its original length.

### BRIEF DESCRIPTION OF DRAWINGS

FIGS. 1A to 1D shows the performance of intrinsically stretchable OECTs on substrates with different oxygen permeabilities (P_{O2}). FIG. 1A shows the structure of OECTs based on conducting polymer PEDOT:PSS channel. FIG. 1B is a schematic diagram of an intrinsically stretchable OECT. FIG. 1C shows the transfer curves of intrinsically stretchable OECTs fabricated on PDMS substrates of different mixing ratios. FIG. 1D is a graph showing the correlation between on/off ratios of the devices and oxygen permeabilities (P_{O2}) of the substrates.

FIGS. 2A to 2C aim to compare the on/off ratios of stretchable OECTs fabricated on different types of stretchable substrates with different P_{O2} values. FIG. 2A shows the chemical structures of different stretchable substrates. FIG. 2B shows the transfer curves of stretchable OECTs fabricated on various substrates. FIG. 2C shows the correlation between the on/off ratios of the devices and the P_{O2} values of corresponding substrates.

FIGS. 3A to 3F serve to benchmark the overall performance of intrinsically stretchable OECTs on low P_{O2} substrates to rigid OECTs on glass. FIG. 3A shows output curves, where the V_{ds} was scanned from 0 V to −0.6 V, and V_{g} was swept from 0 V to 0.8 V with a step of 0.2 V. FIG. 3B shows transfer curves, where V_{ds} was fixed at −0.4 V, and V_{g} was swept from 0 V to 0.8 V. FIG. 3C shows the on/off ratios of intrinsically stretchable OECTs and rigid OECTs with different geometries (width-to-length ratio, W/L). FIG. 3D shows transient response curves, where a pulse of V_{g} of 0.4 V was applied, and the V_{ds} was fixed at −0.2 V. FIG. 3E shows the frequency response profiles of the gₘ, where the V_{ds} was fixed at −0.4 V, and a small signal oscillation of 20 mV was superimposed on a constant V_{g} of 0.1 V to measure the transient gₘ. FIG. 3F show the output I_{ds} of intrinsically stretchable OECT in response to a triangle wave input from 0.05 Hz to 10⁴ Hz.

FIGS. 4A to 4E present the performance of intrinsically stretchable OECTs under strain. FIG. 4A shows the architecture of the intrinsically stretchable OECTs, where PEDOT:PSS films were used as both the electrodes (source, drain and gate) and the channel. FIG. 4B shows transient curves at different strain values, where the V_{g} was swept from 0 V to 0.9 V, and V_{ds} was fixed at −0.4 V. FIG. 4C shows the transfer curves after different strain cycles (50% strain). FIG. 4D shows the comparison of overall performance of the intrinsically stretchable OECTs in this work with previously reported stretchable OECTs, where the x-axel indicates the highest on/off ratios, the y-axel indicates the maximum tolerable strains, and the z-axel indicates the maximum hole mobilities of the channel. FIG. 4E shows the optical microscopic images of PEDOT:PSS films fabricated on different stretchable substrates and under different strain values (0%, 30% and 50% respectively).

FIG. 5 is an overall illustration showing that doping an intrinsically stretchable OECT on a low P_{O2} substrate has the effect of significantly lowering the I_{ds} upon an increasing V_{g}.

FIGS. 6A and 6B compare the on/off ratios of OECT device on different types of thermoplastic polyurethane (TPU). FIG. 6A shows the transfer curves obtained from OECT fabricated on three different types of TPU substrates at V_{ds}=−0.4 v. TPU1 was provided with tightly pack segments, TPU2 is a polyether-based elastomer and TPU3 is a semi-permeable TPU film. FIG. 6B shows the on/off ratios of devices fabricated on three types of TPU substrates and the corresponding P_{O2} of those substrates.

FIGS. 7A and 7B compare the mobility of PEDOT:PSS channel on glass and low P_{O2} TPU substrates. FIG. 7A shows the response of I_{ds} on TPU substrate (left) and on glass substrate (right) at different gate current (I_{g}s) values. A constant V_{ds} of −0.2 V was applied. The channel width is 5 mm, and the channel length is 1.3 mm. FIG. 7B shows the correlation of the derivative of I_{ds} with respect to time as a function of I_{gs}.

FIGS. 8A and 8B compare the mobility of PEDOT:PSS channel on PDMS and TPU substrate. FIG. 8A shows the response of I_{ds} when a series of I_{g}s was applied to stretchable OECT on TPU (left) and PDMS (right). A constant V_{ds} of −0.2 V was applied. The device has a channel width of 5 mm and channel length of 1.3 mm. FIG. 8B shows the correlation of the derivative of I_{ds} with respect to time as a function of I_{gs}.

FIG. 9 shows the transfer curves of rigid OECT (left) and stretchable OECT (right) with different geometries (width-length ratios, W/L). The V_{ds} was −0.4 V, and the width of the device was fixed at 5 mm.

FIG. 10 shows the transfer curve (left) and output curve (right) of fully PEDOT:PSS-based stretchable OECT.

FIG. 11 shows the transfer curves of stretchable OECT at different strain values. The fully stretchable OECT was fabricated on TPU. A solid-state and stretchable gel was used as the stretchable electrolyte. Liquid metal (eutectic gallium-indium, EGaIn) was used to facilitate the probing of the electrodes during the strain test.

FIG. 12 shows the strain-insensitive feature of the stretchable OECT. The left figure shows the transfer curves of the stretchable OECT device being stretched among the 10^{th} to 20^{th} circles, and the right figure is a zoom-in figure.

FIG. 13 shows the change of maximum transconductance (gₘ) of stretchable OECT after multiple strain (50%) cycles.

FIG. 14 is a comparison of the channel mobility at different strain values.

FIG. 15 is a comparison of the resistance change (R/R₀) of the PEDOT:PSS films fabricated on different stretchable substrates.

FIGS. 16A and 16B show the thickness-dependent stretchability profile of PEDOT:PSS films on TPU substrates. FIG. 16A shows the optical images of PEDOT:PSS films of different thicknesses under different strain values (0%, 30% and 50% respectively). The thickness was controlled by changing the spin-coating from 500×30 s rpm to 3000×30 rpm. FIG. 16B shows the strain-resistance profile of PEDOT:PSS films with different thicknesses. The dimension of the PEDOT:PSS films used were 1 cm in length and 0.5 cm in width. The resistance was measured by using liquid metal as the probing electrode.

FIGS. 17A and 17B show the water stability of PEDOT:PSS films on TPU with and without the addition of GOPS. FIG. 17A is a comparison of the resistance change under strain for PEDOT:PSS film before and after adding GOPS. FIG. 17B shows the enhancement of water stability by the addition of GOPs. Samples (with or without GOPS) were immersed in water for up to 7 days. The samples have a width of 1 cm and a length of 0.5 cm.

FIG. 18 shows the cyclic stability of the electromechanical performance of PEDOT:PSS film on TPU. The left figure shows the current variation between 0% and 30% train recorded for 150 strain cycles, and the right figure is a zoom-in image for the strain cycles between 140 and 150. I₀ denotes the initial current and I denotes the real-time recorded current.

FIGS. 19A and 19B show the mechanical profiles of TPU and PDMS elastomers. FIG. 19A shows the stress-strain curve of TPU and PDMS elastomers. FIG. 19B is a comparison of Young's Modulus of PDMS and TPU, where the data is extracted directly from FIG. 19A.

FIG. 20 shows the results of finite element analysis (FEA). The stress distributions of PEDOT:PSS films on TPU (left) and PDMS substrates (right) at 30% strain are shown.

FIG. 21 illustrates the influence of oxygen permeability of the elastomeric substrates on the non-linear response of I_{ds} in stretchable OECTs at a given V_{g} pulse.

FIG. 22A shows the transient responses of PEDOT:PSS OECTs on PDMS substrates with different oxygen permeabilities by hanging the mixing ratios between the monomer and the curing agent. FIG. 22B compares the decay time T of the pulses extracted from FIG. 22A at different V_{g} values.

FIG. 23A is a diagram of the setup of electrochemical analysis. FIG. 23B shows the CV curves of the PEDOT:PSS thin film on PDMS substrates with different oxygen permeabilities controlled by tuning the mixing ratios.

FIG. 24A shows the mimicking of the synaptic behavior of a neuron with OECT. FIG. 24B demonstrates the results of tuning the short-term memory behavior of stretchable OECTs by tuning the oxygen permeabilities of the substrates. Longer retention time of I_{ds} was obtained by decreasing the oxygen permeability from ˜1800 Barrer in PDMS to −1 Barrer in TPU.

FIG. 25 shows the electrochromic phenomenon of PEDOT:PSS on different oxygen permeabilities substrates, where the oxygen permeability of TPU is ˜1 Barrer and that of PDMS is ˜600 Barrer.

### DETAILED DESCRIPTION

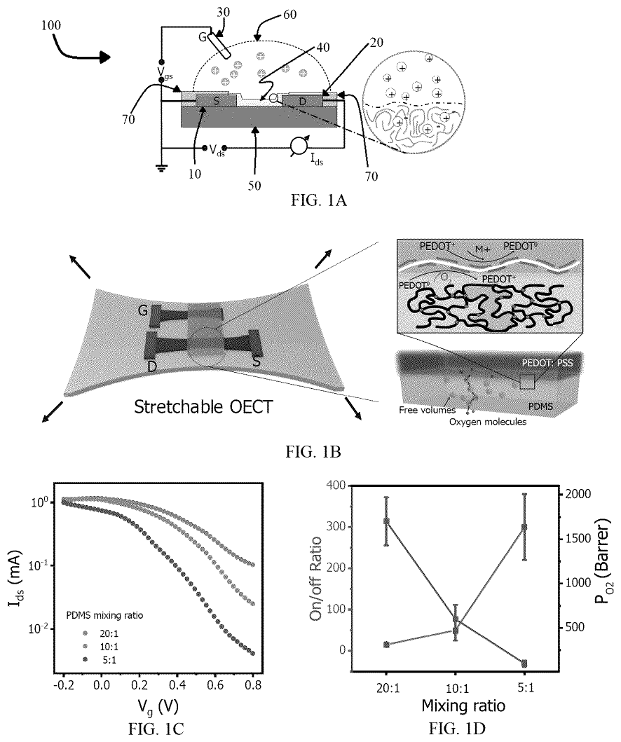

Turning to the drawings in detail, FIG. 1A depicts a device 100 equipped with a stretchable OECT, according to an embodiment. Device 100 includes a source electrode 10, a drain electrode 20 and a gate electrode 30 and a channel 40. The device is positioned on substrate 50, which is a stretchable substrate having a low oxygen permeability. A stretchable solid state electrolyte 60 is cast on the gate electrode 30 and channel 40.

In an embodiment, stretchable substrate 50 is a stretchable elastomer substrate. In particular, the substrate 50 may be a thermoplastic polyurethane; however, other low oxygen permeability substrates may also be used. Such substrates should be stretchable to up to 150% of their original length and have an oxygen permeability of less than 10 Barrer.

In another embodiment, the source electrode 10, drain electrode 20, gate electrode 30 and channel 40 are all formed with a stretchable substance. In particular, the source electrode 10, drain electrode 20, gate electrode 30 and channel 40 are all formed with thin films comprising a poly(3,4-ethylenedioxythiophene)-poly(styrene-sulfonate) mixture. The ratio of poly(3,4-ethylenedioxythiophene) to poly(styrene-sulfonate) in such mixture may be 1:1 to 1:3. The poly(3,4-ethylenedioxythiophene)-poly(styrene-sulfonate) mixture is formed first by stirring a PEDOT:PSS aqueous suspension for 3 minutes and mixing with glycerol (5 v/v. %) and dodecylbenzene sulfonic acid (DBSA) (0.1 V/V. %) with a vortex mixer to increase film conductivity and facilitate the wetting property of films on substrates respectively, and filtering the mixed suspension with a polytetrafluoroethylene membrane (aperture size 0.45 μm) to remove aggregates for further use. In another embodiment, the poly(3,4-ethylenedioxythiophene)-poly(styrene-sulfonate) mixture may further include a (3-glycidyloxypropyl)trimethoxysilane crosslinker.

In yet another embodiment, the device 100 may further comprise a surfactant. In particular, the surfactant may be dodecylbenzene sulfonic acid.

In other embodiment, the solid state electrolyte 60 may be a stretchable ionic gel. In particular, the stretchable ionic gel may be a polyacrylamide-based gel. The polyacrylamide-based gel may be synthesized by (i) dissolving the powder of 15.5 w/w % acrylamide in 16 ml deionized water; (ii) adding 0.01875% w/w % N,N′-methylenebisacrylamide as crosslinker; (iii) adding 1.2 w/w % ammonium persulfide as a photo-initiator; (iv) adding 10 w/w % NaCl to improve ionic conductivity; (v) adding N′-tetramethylethylenediamine at 0.025% weight of the acrylamide as crosslinking accelerator; (vi) transferring the above precursor solution to a dish and cured by exposure to an ultraviolet lamp (365 nm, 5 mW cm⁻²); and (vii) obtaining the stretchable gel after solvent exchange by immersing the cured hydrogel into a bath composing of 85% glycerol, 5% water and 10% NaCl for 2 days.

In another embodiment, reduction of the mobility of the transistor of device 100 is less than 10% when device 100 is stretched up to 110% of its original length. In yet another embodiment, the reduction of the mobility of the transistor of device 100 is less than 25% when device 100 is stretched up to 150% of its original length.

A method for preparing device 100 is also disclosed in another aspect. Firstly, stretchable thin films are formed as the planar gate electrode 30 parallel to the channel 40. Source electrode 10 and drain electrode 20 made of stretchable material are patterned onto a stretchable substrate 50. A stretchable layer 70 is cast on the sub-pattern. The film is annealed to obtain a channel 40; in particular, the annealing is at a temperature of 80-120° C. for a duration of 15-30 minutes. Finally, a stretchable solid state electrolyte 60 is cast on channel 40 and gate 30.

In an embodiment, the stretchable thin films forming source electrode 10, drain electrode 20, gate electrode 30, channel 40 and stretchable layer 70 are formed with a mixture of poly(3,4-ethylenedioxythiophene) and poly(styrene-sulfonate), where the ratio of poly(3,4-ethylenedioxythiophene) and poly(styrene-sulfonate) may be 1:1 to 1:3. In another embodiment, the poly(3,4-ethylenedioxythiophene)-poly(styrene-sulfonate) mixture may further contain a crosslinker. The crosslinker may be (3-glycidyloxypropyl)trimethoxysilane; however, other crosslinkers may be used.

In one embodiment, stretchable substrate 50 is a stretchable elastomer substrate. In particular, the substrate 50 may be a thermoplastic polyurethane; however, other low oxygen permeability substrates may also be used. Such substrates should be stretchable to up to 150% of their original length and have an oxygen permeability of less than 10 Barrer.

In other embodiment, stretchable solid state electrolyte 60 may be a stretchable ionic gel. In particular, the stretchable ionic gel may be a polyacrylamide-based gel.

In yet another embodiment, the device 100 may further comprise a surfactant. In particular, the surfactant may be dodecylbenzene sulfonic acid.

### EXAMPLES

### Example 1: Investigating on/Off Ratios of Elastomers with Different P_{O2} Values

Compared to a rigid device, PEDOT:PSS OECTs fabricated on stretchable elastomers have been suffering from low on/off ratios. Given that the on/off ratio of OECT is mainly dominated by the off-state current (i.e., the de-doping level of the PEDOT:PSS channel) (FIG. 1A), it is hypothesized that hidden parameters exist in the stretchable substrate that can prevent the channel from de-doping, and oxygen permeability is a dominant hidden parameter. As oxygen molecules significantly affect the redox process, and stretchable elastomers being more porous and having the larger free volume in comparison to rigid substrates which in turn provide accessible pathways for oxygen molecules, would increase the oxygen level and prevents PEDOT⁺ from de-doping at the substrate/channel interface. As a result, the oxygen level in a stretchable elastomer could be up to ten times larger than that in the electrolyte, and thus considerably affect the on/off ratio.

To verify the hypothesis, PDMS elastomers with different oxygen permeabilities were prepare by controlling the mixing ratios (FIG. 1C). Before preparing PDMS substrates, cetyltrimethylammonium bromide (CTAB) solution (0.005 M) was prepared and then spun coated on a glass slide as an anti-adhesive layer to ease PDMS peeling-off at the end of the process. In the next step, the base and curing agent of PDMS were mixed. To obtain PDMS with different oxygen permeability, the mixing ratio between base and curing agent ranges from 5:1 (low permeable) to 20:1 (highly permeable). After removing the bubbles under vacuum, the premixed slurry was spin-coated on the glass substrates at 500 rpm for 10 s and 1000 rpm for 30 s. Afterward, the samples were cured at 80° C. for 30 minutes in an oven. Finally, the PDMS substrates were detached from the glass slides for future use.

The OECT was fabricated firstly by patterning source and drain electrodes, followed by a baking process under 120° C. Subsequently, a layer of PDMS was pasted on the top of the source and drain electrodes for insulation.

The correlation between the on/off ratio and the P_{O2} is shown in FIG. 1D. In line with our hypothesis, reducing the P_{O2} from ˜1700 Barrer to ˜100 Barrer significantly reduces the off-state current (FIG. 1C). For example, the off-state current is about 10⁻¹ mA on substrates with P_{O2} of ˜1700, while the value dropped to 5×10⁻³ mA when P_{O2} was reduced to ˜100 (FIG. 1C). As a result, the on/off ratio increases dramatically from 10 to 300 (FIG. 1D) [(I_{ds}(V_{g}=0 V)/I_{ds}(V_{g}=0.8 V)].

### Example 2: Comparing Performances of Transistors Doped on Substrates with Different P_{O2} Values

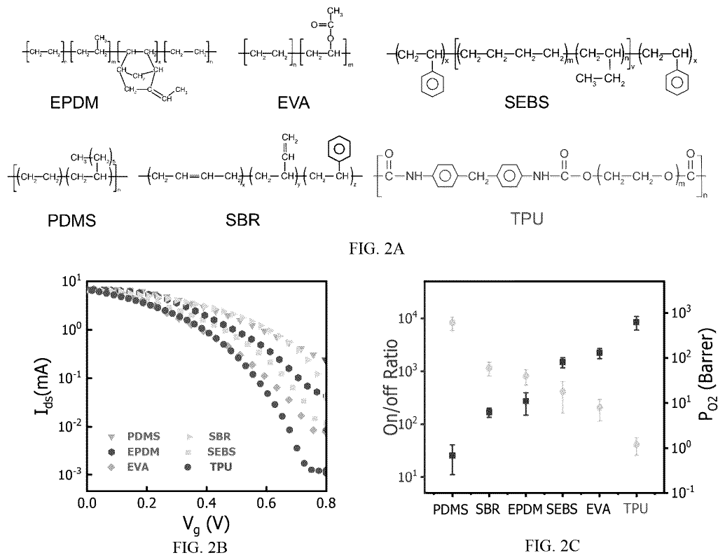

To further verify the results, we subsequently fabricated devices on different types of elastomers, including PDMS, styrene-butadiene rubber (SBR), ethylene propylene diene monomer (EPDM), poly(styrene-ethylene-butylene-styrene) (SEBS), ethylene-vinyl acrylate (EVA) and thermoplastic polyurethane (TPU) (FIG. 2A). The fabrication condition was carefully controlled to let P_{O2} be the major variable parameter.

The elastic substrates were fabricated through a typical solution casting process. TPU grains were dissolved in dimethylformamide (DMF), styrene-ethylene-butylene-styrene block copolymer (SEBS), and styrene-butadiene rubber (SBR) were dissolved in toluene under the heating temperature of 80° C. EVA grains were dissolved in THF at room temperature.

For all elastomer solutions involved, 10 w/w % solutions were prepared by mixing 1 g elastomer with 9 g corresponding solvent. After fully dissolved, the obtained solution was cast on 2.5 cm×7.5 cm glass slides, and the elastic substrates were obtained after drying overnight at room temperature in the fume hood.

The OECT was fabricated firstly by patterning source and drain electrodes, followed by a baking process under 120° C. Subsequently, a layer of PDMS was pasted on the top of the source and drain electrodes for insulation.

To minimize the discrepancy arising from the different film-forming capabilities of PEDOT:PSS on different substrates, PEDOT:PSS film was prepared by firstly spin-coating the mixture suspension on glass slides and then transferred to targeted substrates with a water-soluble tape. To confine the electrolyte, a well was then defined on the top of the channel with a hollow cylinder (diameter of 10 mm). Then a commercial Ag/AgCl electrode was used as a gate electrode, and a 0.1 M NaCl solution was filled in the well as an electrolyte.

The correlation between the on/off ratios and the P_{O2} values of different substrates is shown in FIG. 2B. In agreement with the above results, an inverse relationship was found between the P_{O2} values and the on/off ratios. That is, substrates with lower P_{O2} values tend to harvest higher on/off ratios. In particular, the device fabricated on the TPU substrate of an extremely low P_{O2} of ˜1 showed a record-high on/off ratio of ˜10⁴, which is benchmarkable to the best value reported for rigid device of the same dimension.

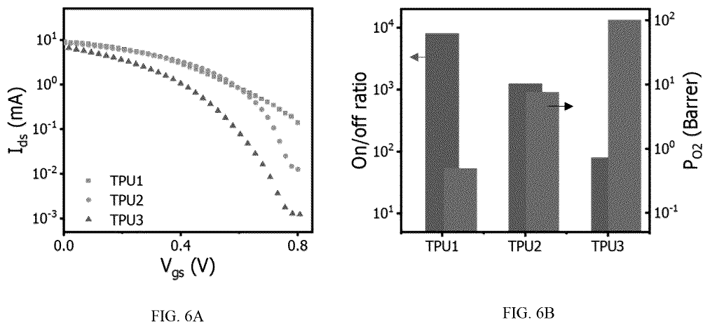

The above results were further verified by comparing on/off ratios of devices fabricated on TPU substrates with different P_{O2} values (FIGS. 6A and 6B).

### Example 3: Benchmarking Performances of Stretchable OECT Devices with OECT Devices on Rigid Substrates

Further investigations were made to compare the overall performance of the stretchable substrates were designed of the same dimension and using the same electrolyte to let the substrate be the major variable parameter.

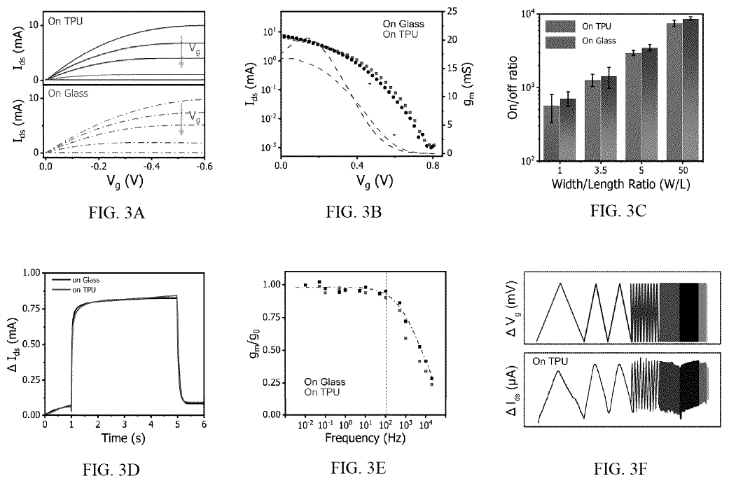

The results are summarized in FIGS. 3A and 3B. As shown, both devices showed similar output and transfer profiles. From the transfer curves, we extracted the corresponding on/off ratios. As shown in FIG. 3C, both devices showed comparable on/off ratios, regardless of the W/L ratios. The on/off ratio reached a high value of ˜104 when the W/L ratio was increased to 50. We further extracted a transconductance (gₘ) of 15 mS (at V_{g}=0.1 V) from those stretchable devices. To the best of our knowledge, this is the highest gm reported so far for intrinsically stretchable PEDOT:PSS OECTs of low P_{O2} with a rigid device fabricated on the glass substrate. To do so, we first compared their steady-state performance, including the output curves and transfer curves.

Next, the transient behavior of stretchable OECTs and rigid OECTs is compared, including their transient response and frequency response profiles. Similar transient response (FIG. 3D) and frequency response (FIG. 3E) curves were obtained for both devices (W/L=50). The gₘ maintained stable values up to 100 Hz, comparable to the rigid device of the same geometry.

According to the Malliaras-Bernards model, the response of OECT can be understood by considering the OECT consisting of two circuits: the ionic one, where ions are transported between the electrolyte and the channel, and the electronic one where holes are transported in the PEDOT:PSS channel between the source and drain. The ionic circuit is governed by the species of ions and the interface between electrolyte and channel. The latter can be estimated by driving the OECT with constant gate current and simultaneously measuring the change of source-drain current regarding response time, and the mobility of PEDOT:PSS on both soft and rigid substrate can be calculated via the below equations (1) to (3):

$${I}_{ds} ( t , {I}_{gs} ) = {I}_{ds 0} - {I}_{gs} ( f + \frac{t}{{τ}_{e}} ) \qquad ( 1 )$$

$$\frac{{dI}_{ds}}{{dI}_{t}} = - \frac{{I}_{gs}}{{τ}_{e}} \qquad ( 2 )$$

$${τ}_{e} = - {L}^{2} / {uV}_{ds} \qquad ( 3 )$$

where t is the time, I_{ds} is the source-drain current before application of I_{gs}, f is a proportionality constant to account for the spatial non-uniformity of the de-doping process, ze is the electronic transit time, L is the channel length of OECT (1.3 mm) and V_{ds} is the source-drain voltage (−0.2 V). u denotes the hole mobility of PEDOT:PSS channel. According to equations (1) and (2), we could calculate the electronic transit time from the linear transient response range of I_{ds} in FIG. 6A. Subsequently, the mobility (u) could be extracted according to equation (3).

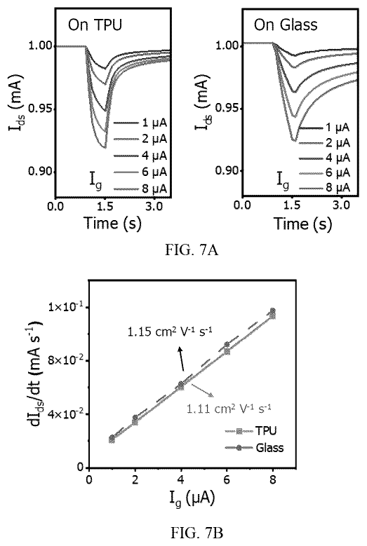

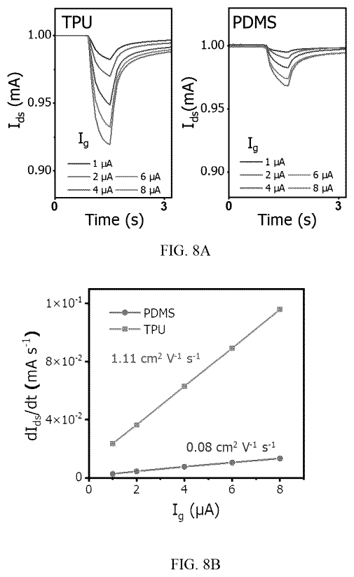

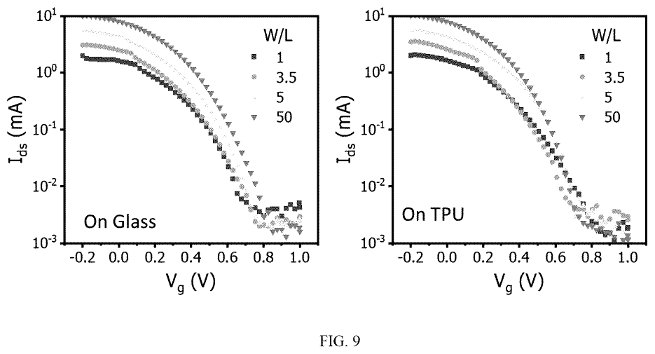

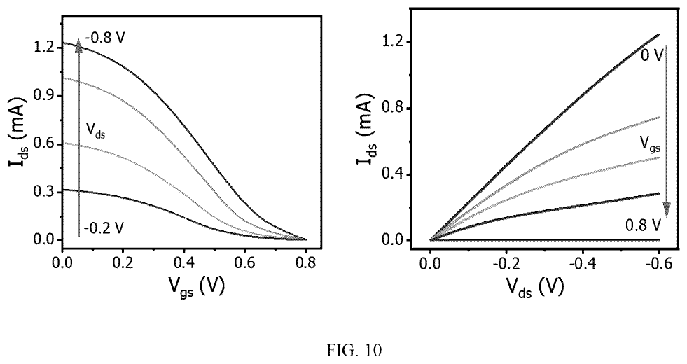

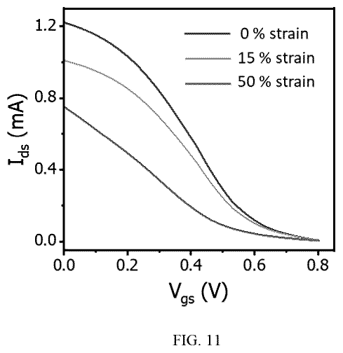

The mobility extracted (FIGS. 7A, 7B, 8A and 8B) is as high as ˜1.1 cm² V⁻¹ s⁻¹, comparable to the rigid device's value (˜1.2 cm² V⁻¹ s⁻¹).

### Example 4: Investigation of the Durability of Stretchable OECT Devices Under Strain

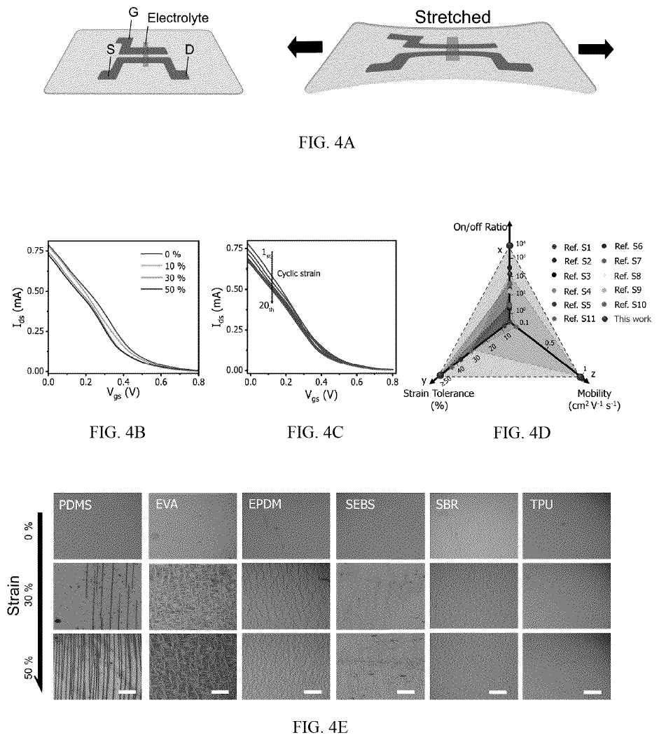

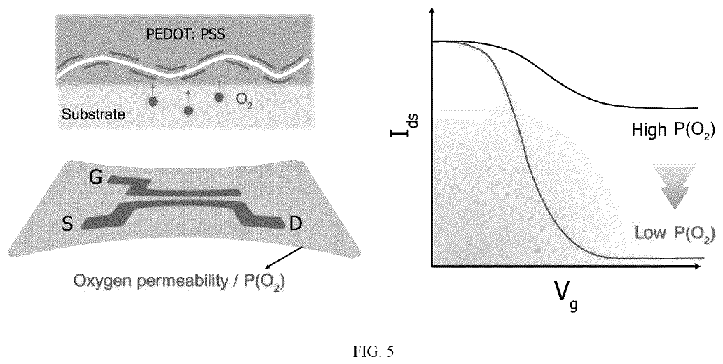

After confirming the high performance, the durability of devices under strain was investigated. To make the device fully stretchable, stretchable PEDOT:PSS thin film was used as the planar gate electrode (FIG. 4A).

In this example, a PEDOT:PSS film was used as the planar gate electrode parallel to the channel. Two parallel PEDOT:PSS stripes were patterned onto the TPU substrate by utilizing the shadow mask. The shadow mask was made of polyamide tape by laser cutting. The mask was attached to the TPU substrate, and then PEDOT:PSS suspension was spun-coated (500 rpm for 15 s and then 1500 rpm for 45 s) on the top of the surface. After lifting off the shadow mask, a patterned PEDOT:PSS channel was obtained after baking the film at 100° C. for 20 minutes. Then, a stretchable ionic gel was used as a stretchable electrolyte by casting on the channel and gate. A liquid metal, eutectic gallium-indium (EGaIn), was utilized to facilitate the probing of the stretchable electrodes under the strain test.

Before the strain test, the device was cyclically stretched to 50% strain three times to stabilize the channel's conductance (FIG. 11).

The transfer curves obtained during the strain test were then shown in FIGS. 4B and 4C.

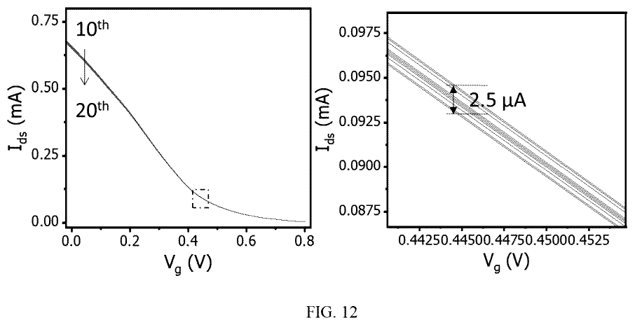

Overall, these transfer curves showed minor changes at different strain values or increased strain cycles (FIG. 12).

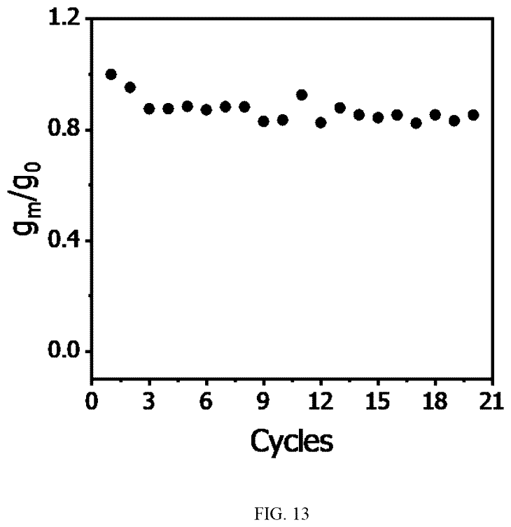

The same trend was recorded for the gₘ (FIG. 13), demonstrating the robustness of the device.

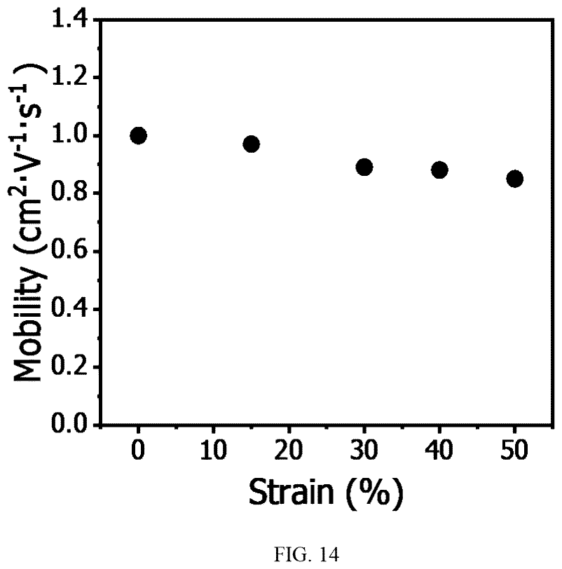

The mobility was maintained at a stable value of around 1 cm² V⁻¹ s⁻¹ before 15% strain. It showed a slight decrease to 0.85 cm² V⁻¹ s⁻¹ at 50% strain (FIG. 14), attributable to the strain-induced expansion of the conductive PEDOT⁺ conjugated polymers, which prohibits the interchain hopping of the holes.

The overall performance of the stretchable OECTs is summarized in FIG. 4D and Table 1 below.

TABLE 1
Benchmarking the performance of stretchable OECTs reported

| | | | Dimension | | | | |
|---|---|---|---|---|---|---|---|
| | | | (mm) | | On/Off | Mobility | Tolerance |
| Literature | Substrate | Channel composition* | W | L | ratio | (cm² V⁻¹ s⁻¹) | strain (%) |
| Ref. S1^{[2]} | PDMS | PEDOT: PSS/EG/ | 0.63 | 0.13 | 3 | N/A | 140 |
| | | DBSA/GOPS/Capstone FS-30 | | | | | |
| Ref. S2^{[3]} | PDMS | PEDOT: PSS/EG/ | 0.4 | 0.25 | −3 | N/A | 38 |
| | | DBSA/GOPS/Capstone FS-30 | | | | | |
| Ref. S3^{[4]} | PDMS | PEDOT: PSS/Gly/ | 4 | 0.01 | 2-10 | N/A | 30 |
| | | DBSA/GOPS/ Capstone FS-30 | | | | | |
| Ref. S4^{[5]} | PDMS | PEDOT: PSS/Gly/ | 4 | 0.01 | 7-150 | N/A | 30 |
| | | DBSA/GOPS/ Capstone FS-30 | | | | | |
| Ref. S5^{[6]} | Skin | PEDOT: PSS/Gly/ | 10 | 1 | 50 | N/A | 5 |
| | | DBSA/GOPS/ Capstone FS-30 | | | | | |
| Ref. S6^{[7]} | SEBS | PEDOT: PSS/ [EMIM][Cl]/ | 1 | 0.25 | 500 | N/A | N/A |
| | | GOPS | | | | | |
| Ref. S7^{[8]} | PDMS | PEDOT: PSS/PEG/Gly/ DBSA/ | 8 | 2 | 50-160 | N/A | 45 |
| | | Capstone FS-30 | | | | | |
| Ref. S8^{[9]} | PDMS | PEDOT: PSS/xylitol/EG/ | N/A | N/A | 170 | 1.16 | 30 |
| | | GOPS/ Capstone FS-30 | | | | | |
| Ref. S9^{[10]} | Acrylic | PEDOT: PSS/EG/DBSA/GOPS | 0.05 | 0.05 | −20 | N/A | 30 |
| | Elastomer | | | | | | |
| Ref. S10^{[11]} | PDMS | PEDOT: Tos/ PEO | 2 | 15 | 45 | N/A | 50 |
| Ref. S11* | PDMS | PEDOT: PSS/Gly/ | 5 | 0.1 | 10-300 | 0.08 | <30 |
| | | DBSA/GOPS | | | | | |
| This work | TPU | PEDOT: PSS/Gly/ | 5 | 0.1 | 8000 | 1.11 | ≥50 |
| | | DBSA/GOPS | | | | | |

As concluded, the device showed a record-high on/off ratio (˜10⁴), high mobility (˜1 cm² V⁻¹ s⁻¹), and stretchability (>50%). This is the first time these high values were harvested in one intrinsically stretchable OECT.

### Example 5: Microscopic Image Inspection of Stretched PEDOT:PSS Films

Finally, the mechanism for the devices' high intrinsic stretchability was investigated by comparing the optical microscopic images of PEDOT:PSS films at different strain values (FIG. 4E).

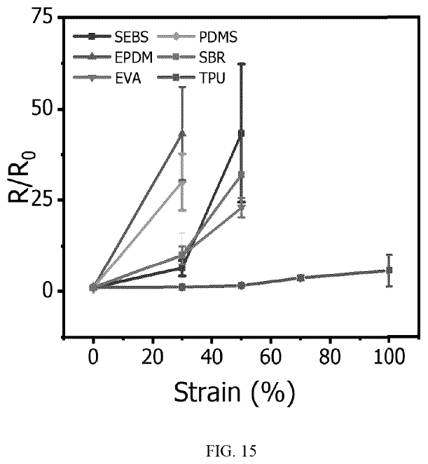

It is noted that PEDOT:PSS films on TPU substrates showed little cracks and maintained a substantial current at 50% strain, while films on other reference substrates showed evident cracking and a considerable current loss at lower strain values (FIG. 15).

It is worth mentioning that the high intrinsic stretchability of PEDOT:PSS films was obtained without the addition of any plasticizers, which are needed for all previous works.

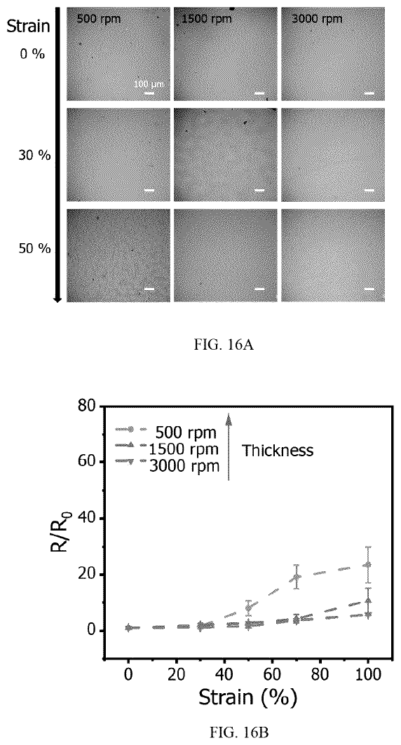

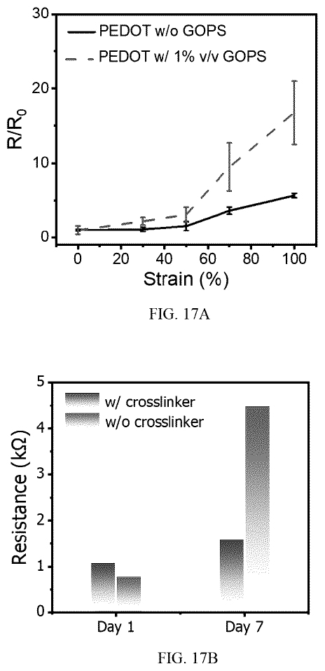

The film's high stretchability was maintained after increasing the film's thickness to 1 μm (FIGS. 16A and 16B) or adding the (3-glycidyloxypropyl) trimethoxy silane (GOPS) crosslinkers (FIGS. 17A and 17B).

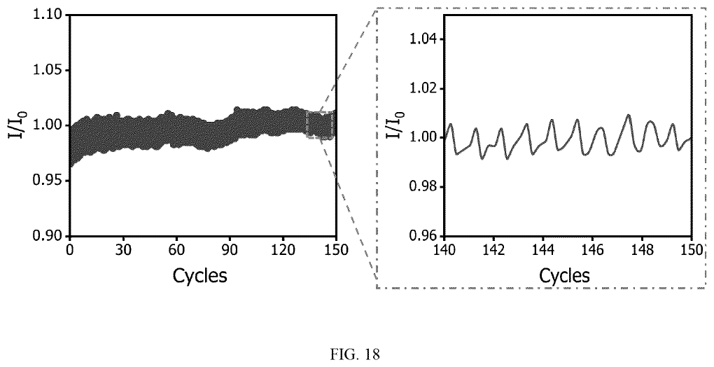

Besides, these films showed minor current loss under cyclic strain tests (FIG. 18).

The high intrinsic stretchability is attributed to the improved matching of Young's modulus between the substrates and the PEDOT:PSS films.

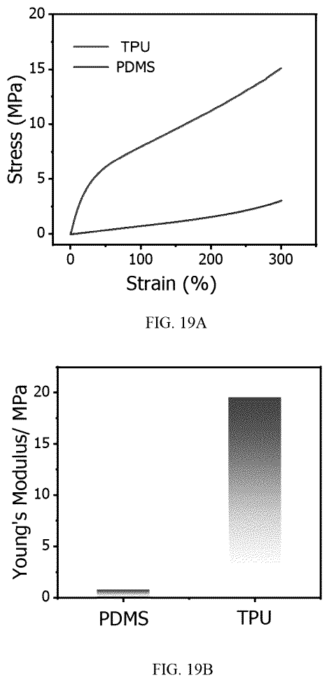

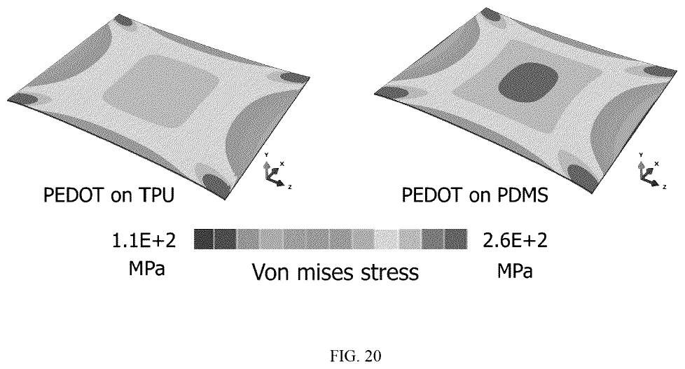

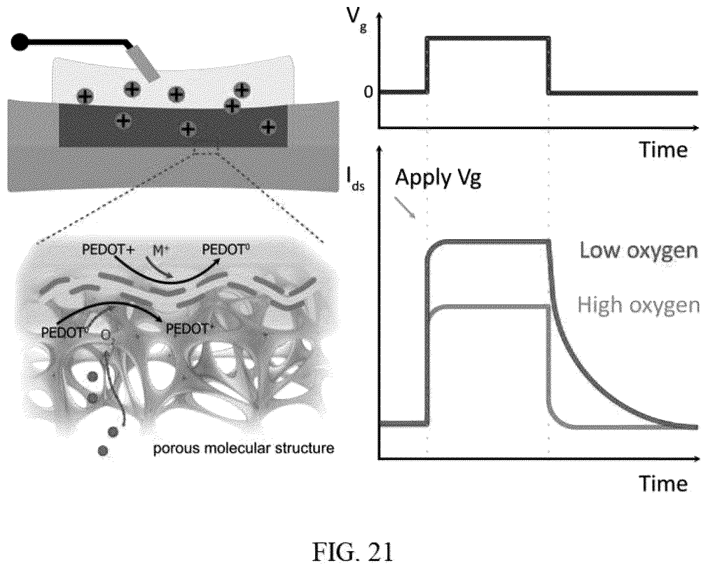

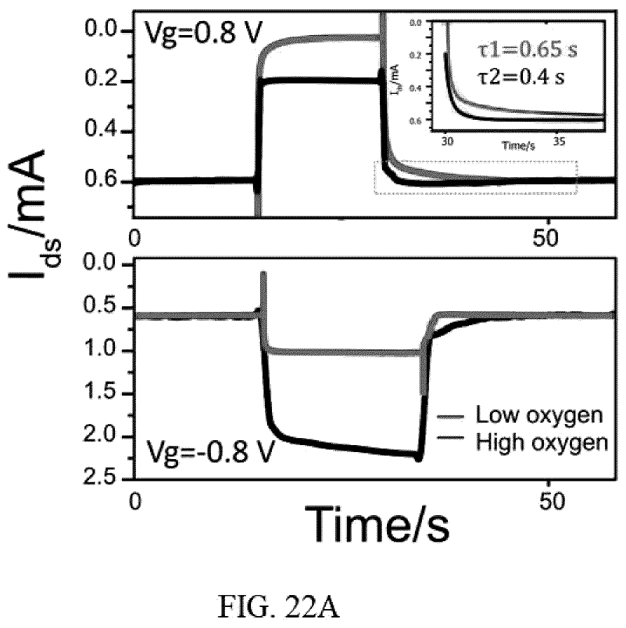

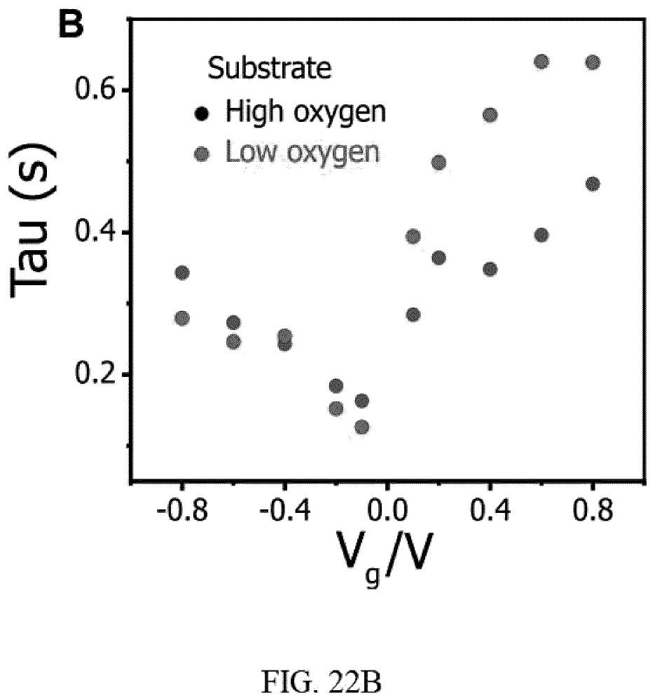

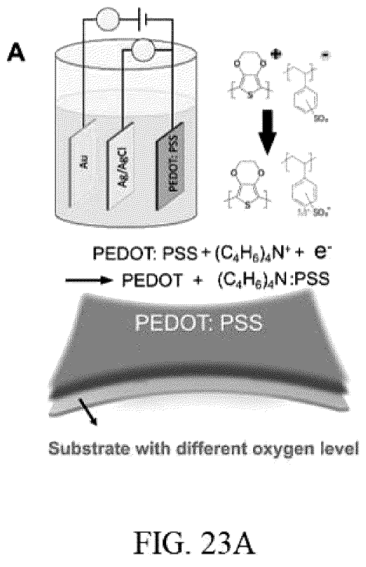

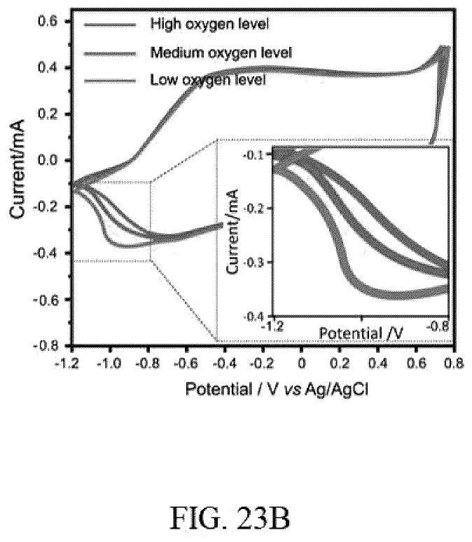

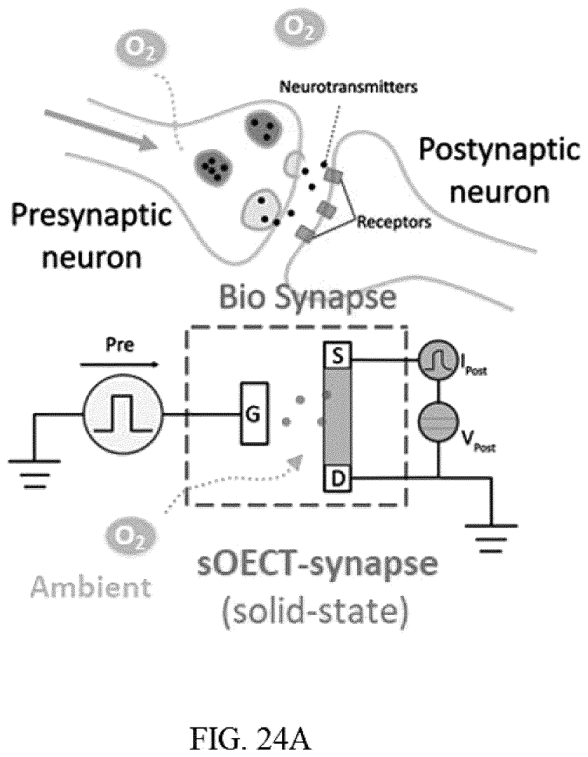

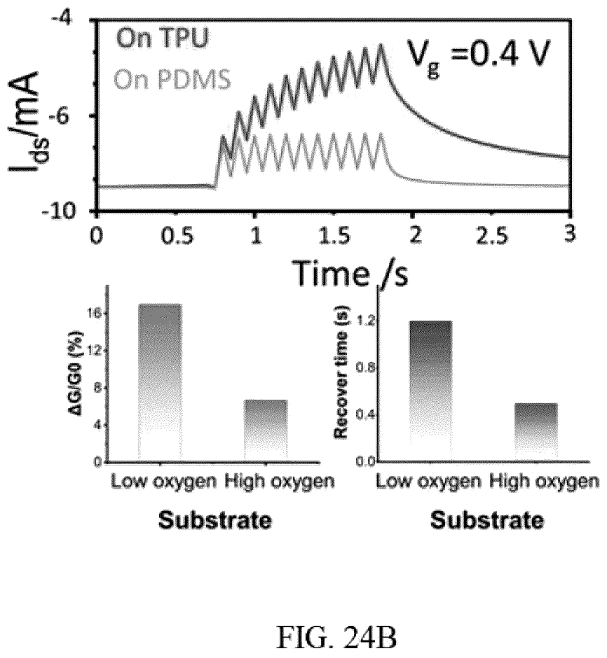

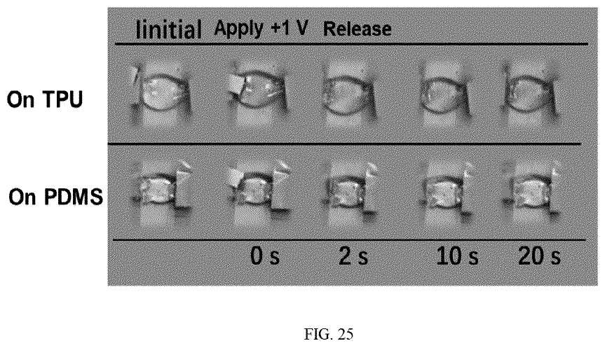

This conclusion is subsequently verified by the Finite Element Analysis (FEA) (FIGS. 19A, 19B and 20), which demonstrates that an improved modulus match of those two layers facilitates the dissipation of the stress and favors the formation of microcracks rather than the large transverse cracks, thus improving the intrinsic stretchability of the device.

The above descriptions are only preferred embodiments of the present invention and are not intended to limit the present invention. Any modification, equivalent replacement and improvement made within the spirit and principle of the present invention shall be included in the protection of the present invention.

### REFERENCES

[1] S. Zhang, E. Hubis, G. Tomasello, G. Soliveri, P. Kumar, F. Cicoira, Chemistry of Materials 2017, 29, 3126.
[2] B. Marchiori, R. Delattre, S. Hannah, S. Blayac, M. Ramuz, Scientific reports 2018, 8, 1.
[3] W. Lee, S. Kobayashi, M. Nagase, Y. Jimbo, I. Saito, Y. Inoue, T. Yambe, M. Sekino, G. G. Malliaras, T. Yokota, Science advances 2018, 4, eaau2426.
[4] S. Zhang, Y. Li, G. Tomasello, M. Anthonisen, X. Li, M. Mazzeo, A. Genco, P. Grutter, F. Cicoira, Advanced Electronic Materials 2019, 5, 1900191.
[5] N. Matsuhisa, Y. Jiang, Z. Liu, G. Chen, C. Wan, Y. Kim, J. Kang, H. Tran, H. C. Wu, I. You, Advanced Electronic Materials 2019, 5, 1900347.
[6] Y. Li, N. Wang, A. Yang, H. Ling, F. Yan, Advanced Electronic Materials 2019, 5, 1900566.
[7] T. D. Nguyen, T. Q. Trung, Y. Lee, N. E. Lee, Advanced Engineering Materials 2021, 2100918.

1. A method for modifying the electrochemical properties, electrochromical properties, non-linearity and synaptic behaviors of an electronic device, comprising:
providing a stretchable substrate selected from one or more of elastomers, hydrogels or hybrid organic-inorganic materials, the stretchable substrate having a selected oxygen permeability of 0.1-50 Barrer; and
forming a stretchable redox-active layer on the substrate, the redox-active layer including one or more of conductive polymers, organic molecules or hybrid organic-inorganic molecules.

2. The method of claim 1, further including depositing one or more electrodes on the stretchable redox-active layer.

3. The method of claim 1, wherein the electronic device is a bioelectronic device in the form of a bioelectrode, a wearable biosensor or a bioelectronic implant.

4. The method of claim 1, wherein the electronic device has a shape of micro-wires, macro-wires, micro-mesh, macro-mesh, film, micro-3D structure and/or macro-3D structure.

5. The method of claim 1, wherein the stretchable substrate is selected from styrene-butadiene rubber, ethylene propylene diene monomer, poly(styrene-ethylene-butylene-styrene), ethylene-vinyl acrylate and/or thermoplastic polyurethane.

6. A stretchable device equipped with an intrinsically stretchable organic electrochemical transistor, comprising:
an OECT comprising a stretchable planar gate electrode, source electrode, drain electrode and channel each comprising a conducting polymer;
a stretchable ionic gel as a solid-state electrolyte cast on the channel and gate; and
a stretchable elastomer substrate;
wherein the width/length ratio of the OECT is 3.5 to 50;
wherein the on/off ratio of the device is at least 10³;
wherein the mobility of the device is at least 0.8 cm²V⁻¹s⁻¹; and
wherein the current loss of the transistor is less than 10% when the device is stretched up to 150% of its original length.

7. The device of claim 6, wherein the stretchable substrate is selected from an elastomer, a hydrogel or a hybrid organic-inorganic stretchable polymer.

8. The device of claim 7, wherein the stretchable substrate is selected from styrene-butadiene rubber, ethylene propylene diene monomer, poly(styrene-ethylene-butylene-styrene), ethylene-vinyl acrylate and/or thermoplastic polyurethane.

9. The device of claim 6, wherein the device has a shape of micro-wires, macro-wires, micro-mesh, macro-mesh, film, micro-3D structure and/or macro-3D structure.

10. The device of claim 6, wherein the conducting polymer is a mixture of poly(3,4-ethylenedioxythiophene) and poly(styrene-sulfonate).

11. The device of claim 10, wherein the ratio of the poly(3,4-ethylenedioxythiophene) to poly(styrene-sulfonate) is 1:1 to 1:3.

12. The device of claim 6, wherein the stretchable ionic gel is a polyacrylamide-based gel.

13. The device of claim 6, wherein the reduction of the mobility of the transistor is less than 10% when the device is stretched up to 110% of its original length.

14. The device of claim 6, wherein the reduction of the mobility of the transistor is less than 25% when the device is stretched up to 150% of its original length.
